(19) United States
(12) Patent Application Publication
(10) Pub. No.: US 2007/0244402 A1
(43) Pub. Date: Oct. 18, 2007

(54) System and method of monitoring physiological signals

(76) Inventors: Brian P. Brockway, Shoreview, MN (US); Vladimir Kovtun, Inver Grove Heights, MN (US); Carlos Ricci, Apple Valley, MN (US); John Arnold, Minneapolis, MN (US); Keith Jasperson, Andover, MN (US); Kathy L. Sherwood, North Oaks, MN (US); Scott Thomas Mazar, Woodbury, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/677,015

(22) Filed: Feb. 20, 2007

Related U.S. Application Data
(60) Provisional application No. 60/774,759, filed on Feb. 17, 2006.

(51) Int. Cl. A61B 5/0402

(52) U.S. Cl. 600/509

(57) ABSTRACT

A physiological signal monitoring and analysis system including an implantable medical device and a signal processor. The implantable medical device is configured to monitor and record sample segments of at least one physiological signal of a patient at time separated recording intervals over a time period. The signal processor configured to measure values of at least one selected characteristic of the at least one physiological signal from the recorded sample segments, to determine trend information representing a trend in the at least one selected characteristic based on the measured values, and to assess a risk of a physiological event to the patient based on the trend information.

Sudden cardiac death (SCD) is a leading cause of death in the United States. The most common cause of SCD is ventricular fibrillation. Ventricular fibrillation is a rapid and disorganized firing of muscle fibers within the ventricular myocardium. During ventricular fibrillation, the ventricles do not contract in an organized manner, no blood is pumped, and blood pressure falls to zero. Because the heart is pumping no blood, patient death may occur within four minutes from the onset of ventricular fibrillation.

One effective treatment for ventricular fibrillation is electrical defibrillation, which applies an electrical shock to the patient's heart. The electrical shock clears the heart of the abnormal electrical activity by depolarizing a critical mass of myocardial cells to allow spontaneous organized myocardial repolarization to resume.

There are two general types of defibrillators; implantable defibrillators and automatic external defibrillators (AED). Implantable defibrillators have the advantage of already being in place when the need for defibrillation arises. One challenge relating to the use of implantable defibrillators is the challenge of accurately identifying patients who are likely to require defibrillation at some point in the future, particularly those patients known to already be at an elevated risk. This can be done by stratifying patients into sudden cardiac death risk groups.

However, conventional methods of assessing SCD risk in at-risk patients typically involve only infrequent “spot checks” of risk and typically do not assess the heart's response to normal physical activities and stresses.

### SUMMARY

One embodiment provides a physiological signal monitoring and analysis system including an implantable medical device and a signal processor. The implantable medical device is configured to monitor and record sample segments of at least one physiological signal of a patient at time separated recording intervals over a time period. The signal processor configured to measure values of at least one selected characteristic of the at least one physiological signal from the recorded sample segments, to determine a trend in the at least one selected characteristic based on the measured values, and to assess a risk of a physiological event to the patient based on the trend information.

### BRIEF DESCRIPTION OF THE DRAWINGS

FIG. 1 is a block diagram illustrating generally one embodiment of a system for monitoring, recording, and analyzing physiological signals of a patient.

FIG. 2 is an example graph hypothetically illustrating a relative risk of sudden cardiac death of an at-risk patient.

FIG. 3 is a block diagram illustrating one embodiment of a signal processor suitable for use with the system of FIG. 1.

FIG. 4 is a diagram illustrating an implantable medical device implanted in a human body.

FIG. 5 is a cross-sectional view of one embodiment of an implantable medical device.

FIG. 6 is a cross-sectional view of the implantable medical device of FIG. 5.

FIG. 7 is a cross-sectional view of the implantable medical device of FIG. 6.

FIG. 8 is an axial view of a lead assembly of an implantable medical device.

FIG. 9 is an isometric view illustrating an implantable medical device implanted in a human body.

FIG. 10 is an isometric view illustrating an implantable medical device implanted in a human body.

FIG. 11 is an isometric view illustrating an implantable medical device implanted in a human body.

FIG. 12 is a transverse cross-sectional view illustrating an implantable medical device implanted in a human body.

FIG. 13 is block diagram of one embodiment of an implantable medical device.

FIG. 14 is a plot of an example electrocardiogram signal.

FIG. 15 is a flow diagram illustrating one embodiment of a process employed by the implantable medical device of FIG. 13.

FIG. 16 is a plot of illustrating features of an electrocardiogram cycle.

FIG. 17 is a block diagram illustrating portions of an embodiment of the signal processor of FIG. 2.

FIG. 18 illustrates an example of a plurality of recorded sample segments of an electrocardiogram signal.

FIG. 19 illustrates an example of a plurality of recorded sample segments of an electrocardiogram signal showing T-wave amplitude alternans.

FIG. 20 illustrates an example of a plurality of recorded sample segments of an electrocardiogram signal showing R-R intervals.

FIG. 21 illustrates an example of a plurality of recorded sample segments of an electrocardiogram signal showing QT intervals.

FIG. 22 illustrates an example of a plurality of recorded sample segments of an electrocardiogram signal showing QRS intervals.

FIG. 23 is a hypothetical time-series of composite values of a measured characteristic of an electrocardiogram signal.

FIG. 24 is a flow diagram illustrating one embodiment of a process employed by the signal processor of FIG. 17.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

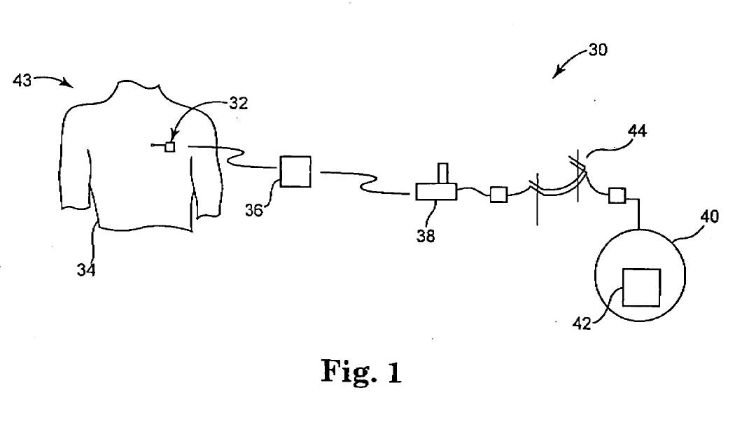

FIG. 1 is block and schematic diagram illustrating generally one embodiment of a system 30 configured to monitor and record sample segments of at least one physiological signal of a patient (including ambulatory patients) at time separated recording intervals over a time period, to measure values of at least one selected characteristic of the physiological signal from the recorded sample segments, and to analyze the measured values to determine and identify a trend in the selected characteristic. System 30 includes an implantable medical device 32 implanted in a body 34, a relay device 36, a base station 38, and a monitoring station 40 including a signal processor 42. In one embodiment, as illustrated, body 34 comprises a body of human patient 43.

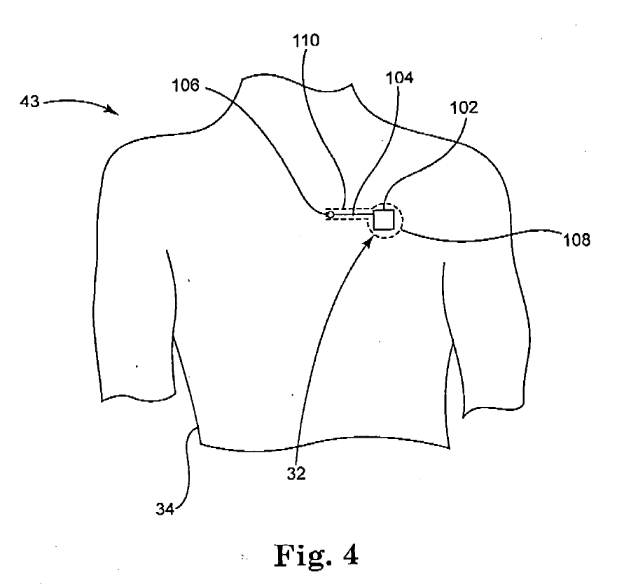

In one embodiment, implantable medical device 32, which is described in greater detail below with respect to FIGS. 4-8, is configured to record sample segments of at least one physiological signal of patient 43 at selectable intervals over a time period. In one embodiment, as will be described in greater detail below, the physiological signal comprises an electrocardiogram (ECG) signal. Implantable medical device 32 is configured to transmit (e.g. wirelessly) the recorded sample segments of the physiological signal to relay device 36 which, in-turn, transmits the recorded sample segments to base station 38. Base station 38 subsequently transmits the recorded sample segments, via a network 44, to monitoring station 40.

In one embodiment, as will be described in greater detail below, signal processor 42 of monitoring station 40 is configured to measure values of the selected characteristic from the recorded sample segments. In one embodiment, based on the measured values, signal processor 42 is configured to determine a trend in the selected characteristic of the physiological signal. In one embodiment, based on the measured values, signal processor 42 is configured to predict a future value of the selected characteristic. In one embodiment, signal processor 42 is configured to assess a risk of patient 43 to a physiological event based on trend information.

It is noted that networks suitable for use as network 44 include the Internet and modem communication via telephone lines, for example. Examples of communications techniques which may be suitable for use with system 30 are described by U.S. Pat. Nos. 5,113,869; 5,336,245; 6,409,674; 6,347,245; 6,577,901; 6,804,559; 6,820,057; and U.S. Patent Application Nos. US2002/0120200 and US2003/0074035.

In one embodiment, as indicated above, the physiological signal monitored and recorded by system 30 comprises an electrocardiogram (ECG) waveform. ECGs are measurements of the electrical activity of the heart. ECGs are reflective of various aspects of the physical condition of the human heart and are employed, for example, to measure the rate and regularity of heartbeats, to detect the presence of damage to the heart, to monitor the effects of drugs, and for providing information to devices used to regulate heartbeats (e.g. defibrillators).

Analysis of electrical cardiac activity can also provide significant insight into the risk of a patient for sudden cardiac death (SCD). Identification of spurious electrical activity with the heart can provide physicians with clues as to the relative cardiac risk presented to the patient. T-wave alternans, in particular, which will be described in greater detail below, is recognized to be associated with electrical instability of the heart and has been recognized as a significant indicator of risk for ventricular arrhythmia and SCD. Other ECG characteristics or features which may be employed to assess the risk of SCD include heart rate variability, heart rate periodicity, and QT-interval, to name a few.

SCD is a leading cause of death in the United States. As such, in order for physicians to provide proper therapy to prevent SCD, it is important to accurately assess the risk of SCD to patients, particularly those patients already known to be at an elevated risk, such as those patients identified as candidates for an implantable cardioverter defibrillator (ICD) under the MADIT II or SCD-HeFT criteria. Such at-risk patients may have already experienced a myocardial infarction (MI) resulting in a deterioration of their myocardium.

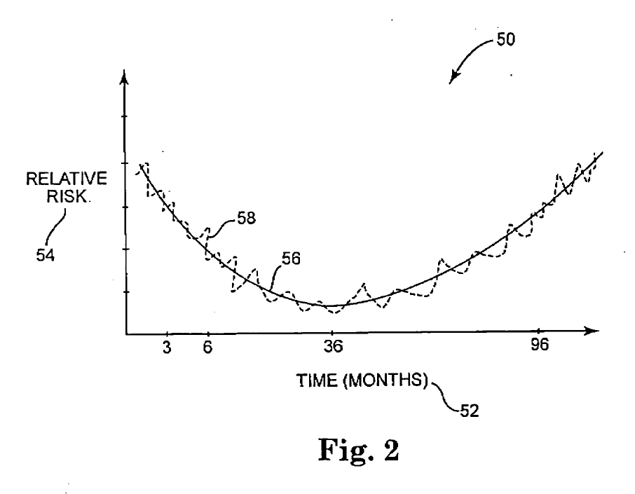

FIG. 2 is graph 50 generally illustrating a hypothetical relative risk over time of an arrhythmic event that could lead to SCD for such a patient. Time (in months) is indicated along x-axis 52, and relative risk is illustrated along y-axis 54. As illustrated by the solid line of curve 56, following an MI, an underlying trend in relative risk initially moves downward. However, there is a tendency of the myocardium to remodel over a period of years and such that the underlying trend in relative risk moves increasingly upward over time such that the patient become increasingly susceptible to SCD. However, over shorter time periods, as illustrated by the dashed line of curve 58, the relative risk of SCD modulates and is highly variable. Such modulations result from highly variable stressors such as blood potassium, neurohormonal signaling (such as increases in BNP (B-type nutriuretic peptide) resulting from elevated LV (left ventricle) filling pressure), coronary vessel spasm, catecholamine releasing events (e.g. psychological stress), and physical activity. Such stressors can vary over the course of minutes or hours.

Conventional methods of assessing SCD risk in at-risk patients, such as those identified as such by MADIT II and SCD-HeFT criteria, typically involve only infrequent “spot checks” of risk (e.g. micro volt TWA is typically performed years), because such checks typically require the patient to visit a healthcare facility and involve time consuming set-up processes, such as application of external electrodes to the patient. Such tests also typically involve simulating physical activity by elevating a patient's heart rate to predetermined level, such as by having the patient walk on a treadmill, for example. However, because of the time varying nature of the risk of SCD to the patient, both in the long term (curve 56) and short term (curve 58), it is difficult to accurately assess the SDC risk of a patient based on such infrequent monitoring. Additionally, such checks do not accurately gauge the heart's response to normal physical activities and stresses.

By recording and wirelessly transmitting recorded sample segments of an ECG signal via implantable medical device 32, system 30 substantially eliminates the need for patient compliance in data collection (e.g. visiting a healthcare facility and connection of external electrodes) and enables the ECG signal of patient 43 to be monitored and recorded while patient 43 is ambulatory and going about normal activities. System 30 also enables monitoring and recording of a patient's ECG signal on a more frequent basis and over longer periods of time than systems and methods requiring a patient to visit a healthcare facility. In one embodiment, for example, system 30 monitors and records sample segments of an ECG on a weekly basis for a year. In another embodiment, system 30 monitors and records sample segments of an ECG on a monthly basis for one or more years. In one embodiment, as will be described in greater detail below, implantable medical device 32 is configured to monitor a heart rate of patient 43, and is configured to record sample segments of an ECG when the heart rate is within a predetermined range.

Additionally, by monitoring and recording ECG sample segments on a frequent basis and over long time periods, system 30 is better able to detect both short term modulations and long term trends in SCD risk. Furthermore, system 30 is better able to monitor cardiac activity in response to both physiologically and psychologically stressful activities in which the patient engages as part of the patient's normal activities. As such, monitoring system 30 enables a more accurate assessment of a patient's SCD risk than conventional methods.

In some embodiments, as will be described in greater detail below, from the recorded ECG sample segments, system 30 is able to measure and record one or more time-series of a characteristic or feature of an ECG waveform, such as TWA, for example. The individual time-series derived from the recorded ECG sample segments can be separately evaluated by a physician, or mathematically combined to form a composite times-series which is analyzed by system 30 to determine and identify a trend in the feature that may correlate with a degree of risk of SCD. In other embodiments, a time-series of one characteristic (e.g. TWA) can be correlated with a time-series of one or more other ECG features (e.g. heart rate variability, QT-interval) to assess a degree of risk of SCD. Correlation of two or more variables is often referred to as multi-variate analysis.

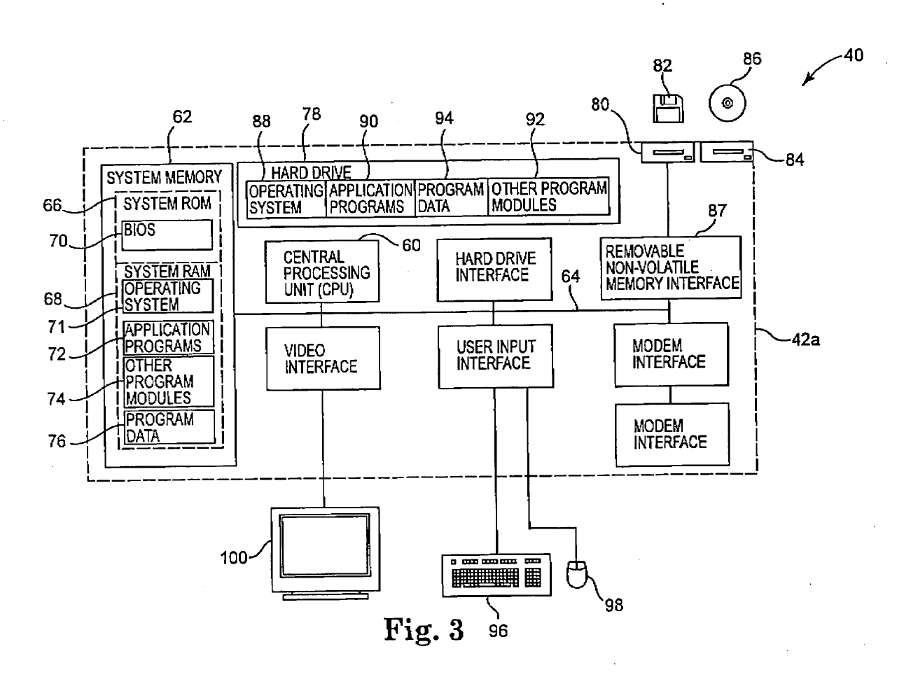

FIG. 3 is a block diagram illustrating one embodiment of signal processor 42, wherein signal processor 42 comprises a computer 42a. In this context, exemplary methods in accordance the present invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

In the embodiment of FIG. 3, computer 42a includes a central processing unit 60, a system memory 62, and a system bus 64 that couples various system components including the system memory to the central processing unit 60. The system bus 64 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 42a typically includes a variety of computer readable media. Computer readable media can be any available media that can be accessed by computer 42a and includes both volatile and nonvolatile media, removable and non-removable media. By way of example, and not limitation, computer readable media may comprise computer storage media and communication media. Computer storage media includes volatile and nonvolatile removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 42a. Communication media typically embodies computer readable instructions, date structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

System memory 62 includes computer storage media in the form of volatile and/or nonvolatile memory such as ROM 66 (read only memory) and RAM 68 (random access memory). A basic input/output system 70 (BIOS), containing the basic routines that help to transfer information between elements within computer 42a, such as during start-up, is typically stored in ROM 66. RAM 68 typically contains data and/or program modules that are immediately accessible to and/or presently being operated on by processing unit 60. In one embodiment, computer 42a includes an operating system 70, application programs 72, other program modules 74, and program data 76.

Computer 42a may also include other removable/non-removable, volatile/nonvolatile computer storage media. For example, in one embodiment, computer 42a includes a hard disk drive 78 that reads from or writes to non-removable, nonvolatile magnetic media, a magnetic disk drive 80 that reads from or writes to a removable, nonvolatile magnetic disk 82, and an optical disk drive 84 that reads from or writes to a removable, nonvolatile optical disk 86 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. In one embodiment, as illustrated by FIG. 3, hard disk drive 78 is connected to system bus 64 through an appropriate interface. Magnetic disk drive 80 and an optical disk drive 84 are connected to system bus 64 by a removable memory interface 87.

The drives and their associated computer storage media discussed above with respect to FIG. 3 provide storage of computer readable instructions, data structures, program modules and other data for the computer 42a. In the embodiment of FIG. 3, for example, hard disk drive 78 is illustrated as storing an operating system 88, application programs 90, other program modules 92, and program data 94. Note that these components can either be the same as or different from operating system 70, application programs 72, other program modules 74, and program data 76. Operating system 88, application programs 90, other program modules 92, and program data 94 are given different numbers here to illustrate that, at a minimum, they are different copies.

A user may enter commands and information into the computer 42a through input devices such as a keyboard 96 and pointing device 98, commonly referred to as a mouse, trackball or touch pad. Other input devices (not shown) may include a microphone, joystick, game pad, satellite dish, scanner, or the like. These and other input devices are often connected to the processing unit 60 through a user input interface that is coupled to the system bus 64, but may be connected by other interface and bus structures, such as a parallel port, game port or a universal serial bus (USB). A display 100 or other type of display device is also connected to the system bus 64 via a video interface. In addition to the monitor, computers may also include other peripheral output devices such as speakers and printers, which may be connected through an output peripheral interface.

FIG. 4 is a plan view illustrating one embodiment of implantable medical device 32 implanted in body 34 of patient 43. In the embodiment of FIG. 4, implantable medical device 32 comprises a housing 102, a lead body 104, and a remote electrode 106. Implantable medical device 32 is configured to detect a voltage difference between remote electrode 106 and housing 102, wherein the voltage difference is representative of a physiological signal of patient 43. In one embodiment, as will be described in greater detail below, the voltage difference is representative of an electrocardiogram signal. In one embodiment, implantable medical device 32 is further configured to selectively and digitally record this voltage difference.

In other embodiments, implantable medical device 32 is configured to provide a therapeutic function. Examples of therapeutic functions include pacing, defibrillation, and cardioversion. With reference to FIG. 4, it will be appreciated that housing 102 is disposed in a pocket 108 and that remote electrode 106 is disposed in a channel 110, wherein pocket 108 and channel 110 are each formed in the tissue of body 34. In other embodiments, pocket 108 and channel 110 are formed within a pre-selected implant site inside human body 34.

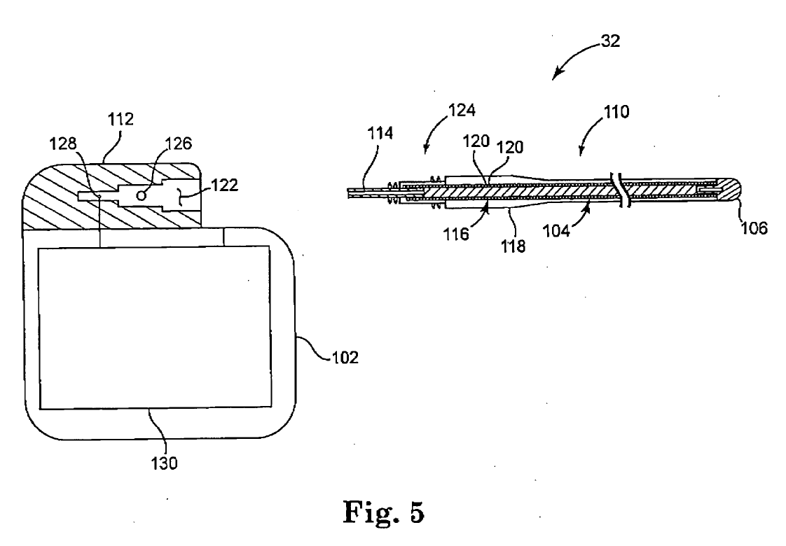

FIG. 5 is a cross-sectional view illustrating one embodiment of implantable medical device 32. Implantable medical device 32 includes conductive housing 102 and a lead assembly 110, with conductive housing 102 including a header 112. Lead assembly 110 includes remote electrode 106 and a connector pin 114. Remote electrode 106 and connector pin 114 are mechanically coupled to one another by lead body 104 of lead assembly 110. Lead body 104 comprises a conductor 116 and an outer sheath 118. In some embodiments, outer sheath 118 comprises a flexible material. Examples of flexible materials that may be suitable in some applications include silicone rubber and polyurethane.

In the embodiment of FIG. 5, remote electrode 106 and connector pin 114 are electrically connected to one another by conductor 116. In the embodiment of FIG. 5, conductor 116 comprises a plurality of coiled filars 120. Conductor 116 may comprise, for example, one or more filars wound in a generally helical shape. For example, conductor 116 may comprise four helically wound filars. Remote electrode 106 may comprise various materials without deviating from the spirit and scope of the present invention. Examples of materials that may be suitable in some applications include stainless steel, Elgiloy, MP-35N, titanium, gold and platinum. Remote electrode 106 may also include a coating. Examples of coatings that may be suitable in some applications include carbon black, platinum black, and iridium oxide.

Header 112 defines a socket 122 that is dimensioned to receive a connecting portion 124 of lead assembly 110. Remote electrode 106 may be detachably coupled to conductive housing 102 by inserting connecting portion 124 of lead assembly 110 into socket 122. In the embodiment of FIG. 5, a set screw 126 is disposed in a threaded hole defined by header 112. Set screw 126 may be used to selectively lock connecting portion 124 of lead assembly 110 in socket 122. An electrical contact 128 is also shown in FIG. 5. Electrical contact 128 may make contact with connector pin 114 when connecting portion 124 of lead assembly 110 is disposed in socket 122. In this way remote electrode 106 can be electrically connected to an electronics module 130 of implantable medical device 32 via connector pin 114 and conductor 116.

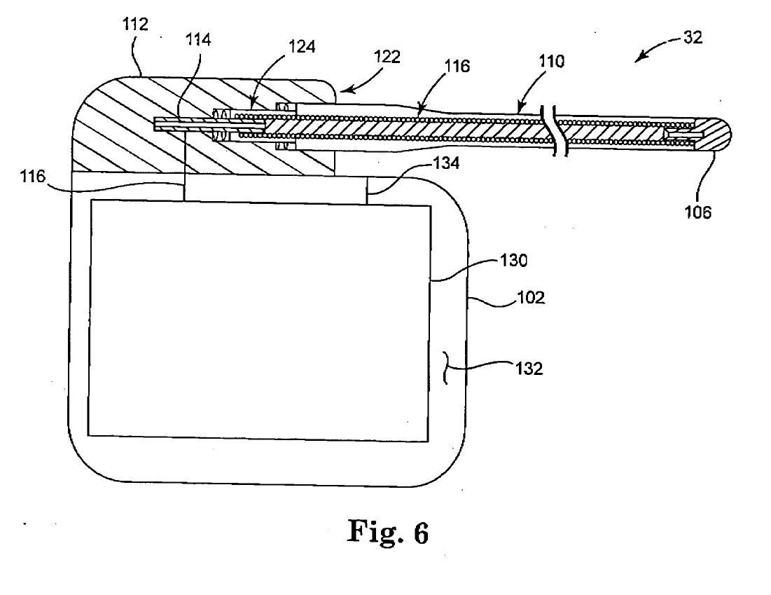

FIG. 6 is an additional cross sectional view of implantable medical device 32 illustrating connecting portion 124 of lead assembly 110 disposed in socket 122 defined by header 112. In the embodiment of FIG. 6, implantable medical device 32 includes electronics module 130 which is disposed within a cavity 132 defined by conductive housing 102. With reference to FIG. 6, it will be appreciated that electronics module 130 is electrically connected to remote electrode 106 via pin 114 and conductor 116. With continuing reference to FIG. 6, it will also be appreciated that electronics module 130 is electrically connected to conductive housing 102 by a wire 134.

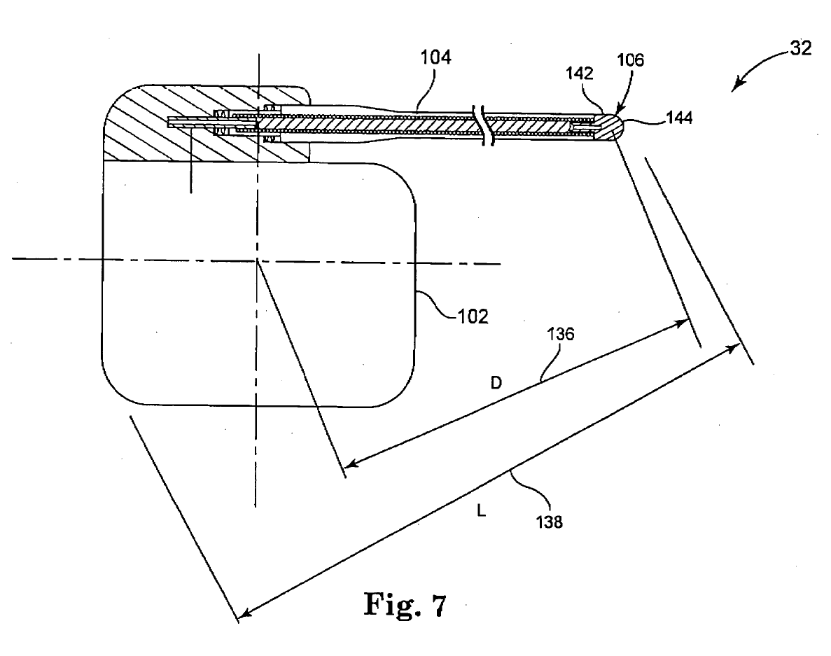

FIG. 7 is an additional cross sectional view of implantable medical device 32. With reference to FIG. 7, it will be appreciated that lead body 104 separates remote electrode 106 and conductive housing 102 by a center-to-center distance D, as indicated at 136. In some embodiments, distance D 136 is configured to be relatively large so that a voltage differential between conductive housing 102 and remote electrode 106 is relatively large. In some embodiments, distance D 136 is greater than about four centimeters and less than about ten centimeters. In some embodiments, distance D 136 is greater than about five centimeters and less than about seven centimeters.

With continuing reference to FIG. 7, it will be appreciated that implantable medical device 32 has an overall length L, as indicated at 138. In some embodiments, overall length L 138 is configured so that conductive housing 102, remote electrode 106, and lead body 104 will all be received in an implant site overlaying one half of a rib cage of a human body. In some embodiments, overall length L 138 is greater than about four centimeters and less than about thirteen centimeters. In some embodiments, overall length L 138 is greater than about five centimeters and less than about ten centimeters.

Conductive housing 102 may comprise various materials without deviating from the spirit and scope of the present invention. Examples of materials that may be suitable in some applications include stainless steel, Elgiloy, MP-35N, titanium, gold and platinum. Conductive housing 102 may also comprise a conductive coating. Examples of conductive coatings that may be suitable in some applications include carbon black, platinum black, and iridium oxide. In the embodiment of FIG. 7, conductive housing 102 is free of insulating coatings so that the entire outer surface of conductive housing 102 is available to make electrical connection with body tissue. Embodiments of the present invention are possible in which a portion of conductive housing 102 is covered with an insulating coating, for example, PARYLENE.

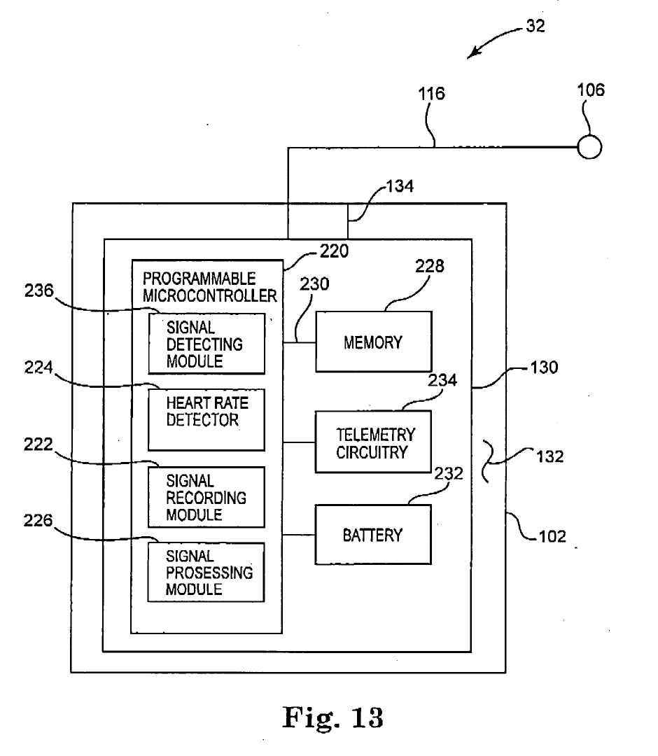

In the embodiment of FIG. 7, remote electrode 106 comprises a generally cylindrical body portion 142 having a generally circular lateral cross section. With reference to FIG. 13, it will be appreciated that remote electrode 106 also comprises a general rounded tip portion 144. In the embodiment of FIG. 13, tip portion 144 has a generally hemispherical shape.

With reference to FIG. 13, it will be appreciated that remote electrode 106 and lead body 104 are both free of anchors. In some applications, providing a remote electrode that is free of anchors may facilitate removal of the remote electrode from the human body. Additionally, providing a lead body that is free of anchors may facilitate removal of the lead from the human body.

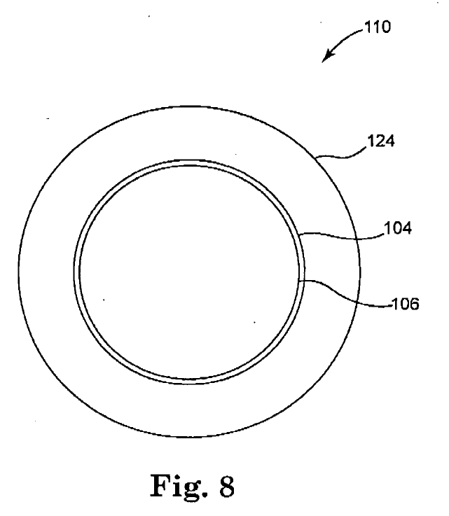

FIG. 8 is an axial view illustrating one embodiment of lead assembly 110. In one embodiment, as illustrated by FIG. 8, remote electrode 106, lead body 104, and connecting portion 124 are generally circular in cross section. In some applications, providing remote electrode 106 having a circular transverse cross-section may facilitate removal of the remote electrode from the human body. Additionally, providing a lead body having a circular transverse cross-section may facilitate removal of the lead from the body.

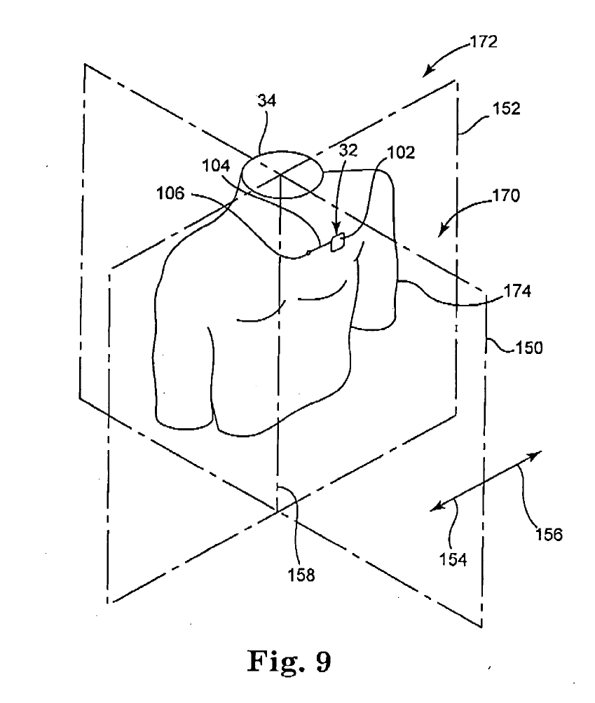

FIG. 9 is an isometric view showing a portion of human body 34 with implantable medical device 32 implanted therein. A central sagital plane 150 and a frontal plane 152 are shown intersecting human body 34. In the embodiment of FIG. 9, central sagital plane 150 and frontal plane 152 intersect one another at a median axis 158 of human body 34. With reference to FIG. 9, it will be appreciated that central sagital plane 150 bisects human body 34 into a right half 154 and a left half 156. Also with reference to FIG. 9, it will be appreciated that frontal plane 152 divides human body 34 into an anterior portion 170 and a posterior portion 172. In the embodiment of FIG. 9, central sagital plane 150 and frontal plane 152 are generally perpendicular to one another.

With reference to FIG. 9, it will be appreciated that implantable medical device 32 is implanted in tissue proximate a left arm 174 of human body 34. As described above, implantable medical device 100 comprises housing 102, remote electrode 106 and lead body 104 that mechanically couples remote electrode 106 to housing 102.

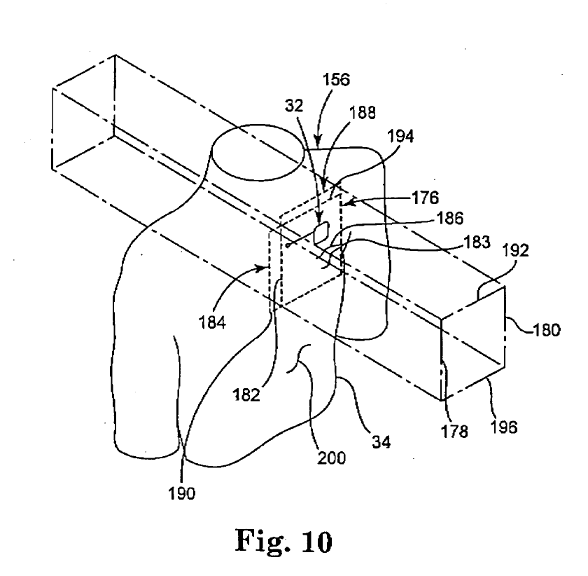

FIG. 10 is an isometric view showing a left implant site 176 disposed in the left half 156 of human body 32 shown in FIG. 9. With reference to FIG. 10, it will be appreciated that implantable medical device 32 is disposed in left implant site 176 as defined by reference to the plurality of planes. A first sagital plane 178 is shown contacting a left-most extent 182 of a sternum 184 of human body 34. A second sagital plane 180 is shown contacting a left-most extent 183 of a rib cage 186. In the embodiment of FIG. 10, left implant site 176 extends laterally between first sagital plane 178 and second sagital plane 180. A superior transverse plane 192 is shown contacting a lower surface 194 of a left clavicle 188 of human body 34. An inferior transverse plane 196 is shown contacting a lower extent 190 of sternum 184. In the embodiment of FIG. 10, left implant site 176 extends between superior transverse plane 192 and inferior transverse plane 196. Some methods in accordance with the present invention include the step of implanting implantable medical device 32 within left implant site 176. In some methods in accordance with the present invention, implantable medical device 32 is implanted between the skin 200 of human body 34 and a front extent of rib cage 186.

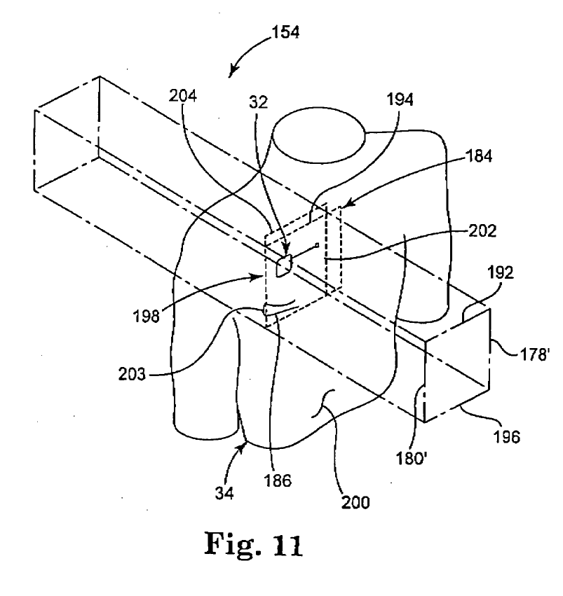

FIG. 11 is an isometric view showing a right implant site 198 disposed in the right half 154 of the human body 34 shown in FIG. 10. With reference to FIG. 11, it will be appreciated that implantable medical device 32 is disposed in the right implant site 198. As shown in FIG. 11, right implant site 198 may be defined by reference to the plurality of planes. A first sagittal plane 178′ is shown contacting a right-most extent 202 of sternum 184 of human body 34. A second sagittal plane 180′ is shown contacting a right-most extent 203 of a rib cage 186. In the embodiment of FIG. 11, right implant site 198 extends laterally between first sagittal plane 178′ and second sagittal plane 180′. A superior transverse plane 192 is shown contacting a lower surface 194 of a right clavicle 204 of human body 34. An inferior transverse plane 196 is shown contacting a lower extent of sternum 184. In the embodiment of FIG. 11, right implant site 198 extends between superior transverse plane 192 and inferior transverse plane 196. Some methods in accordance with the present invention include the step of implanting implantable medical device 32 within right implant site 198. In some methods in accordance with the present invention, implantable medical device 32 is implanted between the skin 200 of human body 34 and a front extent of rib cage 186.

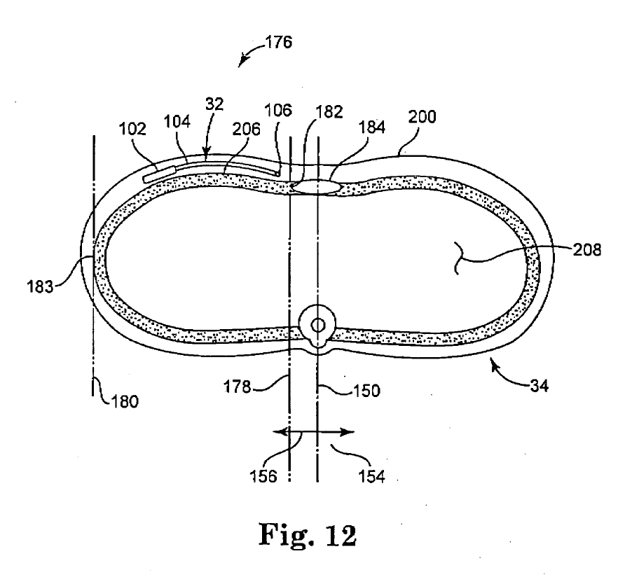

FIG. 12 is a transverse cross-sectional view of human body 34 with implantable medical device 32 implanted therein. Skin 200 and rib cage 186 of human body 34 are visible in this cross-sectional view. With reference to FIG. 12, it will be appreciated that implantable medical device 32 is disposed in a left implant site 176 of human body 34. Central sagital plane 150 is also shown in FIG. 12. With reference to FIG. 12, it will be appreciated that central sagital plane 150 bisects rib cage 186 into right half 154 and left half 156. With reference to FIG. 12, it will be appreciated that left implant site 156 generally overlays left half 156 of rib cage 186.

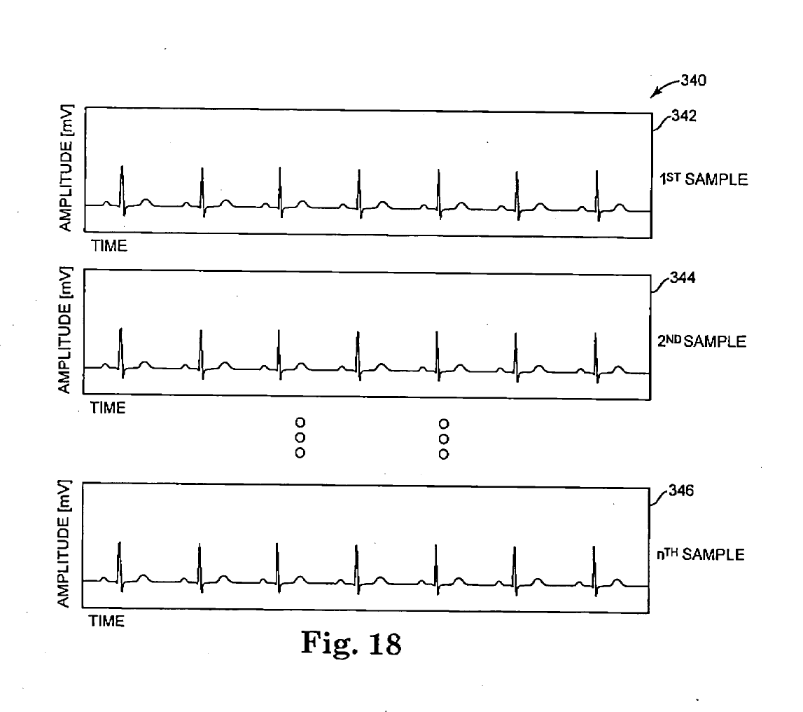

With reference to FIG. 12, it will be appreciated that implantable medical device 32 is disposed between skin 200 of human body 34 and frontal extent 206 of rib cage 186 of human body 34. In the embodiment of FIG. 12, left implant site 176 extends between first sagittal plane 178 and second sagittal plane 180. In FIG. 12, first sagittal plane 178 is shown contacting left-most extent 182 of sternum 184 of human body 34. Also in FIG. 12, second sagittal plane 180 is shown contacting left-most extent 182 of rib cage 186. In the embodiment of FIG. 12, as described above, implantable medical device 32 includes housing 102, lead body 104, and remote electrode 106. In FIG. 12, lead body 104 is shown assuming a generally curved shape. In some embodiments, lead body 104 has sufficient lateral flexibility to allow lead body 104 to conform to the contour of left implant site 176. In other embodiments, lead body 104 has sufficient lateral flexibility to allow lead body 104 to flex in compliance with muscle movements of human body 34. With reference to FIG. 18, it will be appreciated that lead body 104 is disposed outside of a thoracic cavity 208 of human body 34. Accordingly, it will be appreciated that lead body 104 does not extend into a cavity of the heart of human body 34.

FIG. 13 is a block diagram illustrating one embodiment of implantable medical device 32. Implantable medical device 32 includes conductive housing 102 defining cavity 132. In the embodiment of FIG. 13, implantable medical device 32 includes electronics module 130 that is disposed within cavity 132 defined by conductive housing 102. With reference to FIG. 13, it will be appreciated that electronics module 130 is electrically connected to conductive housing 102 by wire 134. Conductive housing 102 may act as a return electrode. In the embodiment of FIG. 13, remote electrode 106 is connected to electronics module 130 by conductor 116.

In the embodiment of FIG. 13, electronics module 130 of implantable medical device 32 comprises a controller 220. Controller 220 may comprise a microprocessor, or equivalent control circuitry and may further include RAM or ROM memory, logic and timing circuitry, state machine circuitry, and I/O circuitry. Controller 200 is capable of monitoring and processing input signals (e.g., data) as controlled by a program code stored in a designated block of memory.

In the embodiment of FIG. 13, controller 220 includes various modules that may be implemented in hardware as part of the controller 220, or as software/firmware instructions programmed into the device and executed on the controller 220 during certain modes of operation. Controller 220 of FIG. 13 includes a signal recording module 222 that may be used to coordinate the recording of selected physiological signals. Controller 220 further includes a heart rate detector 224 that may be used, for example, to determining desirable times to record a physiological signal. A signal processing module 226 is also shown in FIG. 13. Signal processing module 226 may be used to analyze signals that have been recorded by implantable medical device 32.

Controller 220 is coupled to a memory 228 by a suitable data/address bus 230, wherein programmable operating parameters used by the controller 220 may be stored and modified, as required, in order to customize the operation of the implantable medical device 32 to suit the needs of a particular patient. Implantable medical device 32 additionally includes a battery 232 that provides operating power for electronics module 130. Battery 232 is capable of operating at low current drains for long periods of time. Electronics module 130 further includes a telemetry module 234 configured to transmit signals to and receive signals from a receiver, such as relay device 36, external to body 34. In one embodiment, a signal detecting module 236 is configured to detect a voltage difference between conductive housing 102 and remote electrode 106.

In operation, as described briefly above, implantable medical device 32 is configured to monitor and selectively record sample segments the voltage difference between conductive housing 102 and remote electrode 106, which is representative of a physiological signal of body 34 of patient 43, and to transmit the recorded sample segments to monitoring station 40 via relay device 36 and base station 38. In one embodiment, each of the recorded sample segments has a predetermined sample duration. A wide range of sample durations is possible. For example, in one embodiment, each of the recorded sample segments has a sample duration of 15 seconds. In another embodiment, for example, each of the recorded sample segments has a sample duration of 30 seconds. In other embodiments, the sample duration may be less than 15 seconds. In yet another embodiment, each recorded sample has a duration of 10 minutes. It is noted that, in some instance, the sample duration may be limited by short-term memory constraints of implantable medical device 32.

In one embodiment, implantable medical device 32 is configured to record sample segments at predetermined recording intervals. For example, in one embodiment, medical device 32 is configured to record a sample segment of the physiological signal at twenty-four hour intervals. In one embodiment, medical device 32 is configured to record a sample segment of the physiological signal at one week intervals. In one embodiment, medical device 32 is configured to record a sample segment of the physiological signal at monthly intervals (e.g. 30-day intervals). Any number of other recording intervals are possible.

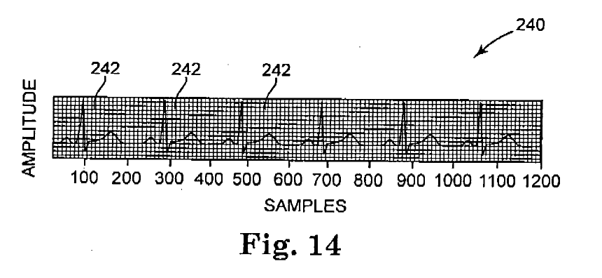

In one embodiment, as mentioned earlier, the voltage difference between conductive housing 102 and remote electrode 106 comprises an ECG signal of patient 43. FIG. 14 is a graph illustrating a recorded sample segment of an ECG signal 240, such as that which may be recorded by implantable medical device 32, with ECG signal 240 comprising a series of cycles or beats 242. As recorded by implantable medical device 32, the recorded sample segment of ECG signal 240 comprises a series of digital data points, wherein each data point includes a sample number, which relates to a time parameter (e.g. at a 1 kHz sampling rate, each sample represents approximately 1 millisecond), and an amplitude (e.g. a voltage level, such as a microvolt level).

In one embodiment, rather than recording at predetermined intervals, heart rate detector 224 monitors the heart rate of patient 43 and implantable medical device 32 records a sample segment of the ECG signal when the heart rate is within a predetermined range. In one embodiment, for example, implantable medical device 32 records a sample segment of the ECG signal when the heart rate is within a range from 92-to-115 beats per minute (bpm) inclusive.

In one embodiment, rather than recording a sample segment of the ECG signal at a recording interval, implantable medical device 32 is configured to record and retain a “best” ECG sample segment during a recording interval based on the heart rate of patient 43. For example, in a scenario where the recording interval comprises a one-week interval, implantable medical device 32 records a present sample segment of the ECG signal when heart rate detector 224 indicates that the heart rate is within the predetermined range. The present sample segment is then compared to a best sample segment which was previously recorded. If the present sample segment is determined to be superior to the best sample segment, the present sample segment becomes the new best sample segment. Conversely, if the previously recorded best sample segment is determined to be superior to the present sample segment, the previously recorded best sample segment remains as the best sample segment. Upon expiration of the recording interval, which in the example is a one-week period, the best sample segment is transmitted to monitoring station 40. The above described process is repeated for each recording interval.

In one embodiment, the comparison is based on the heart rate and a stability of the heart rate. For example, in one embodiment, the best sample segment is the recorded sample segment having the highest heart rate which is within the predetermined range and which is deemed to be stable. In one embodiment, signal processing module 226 is configured to perform the comparison process.

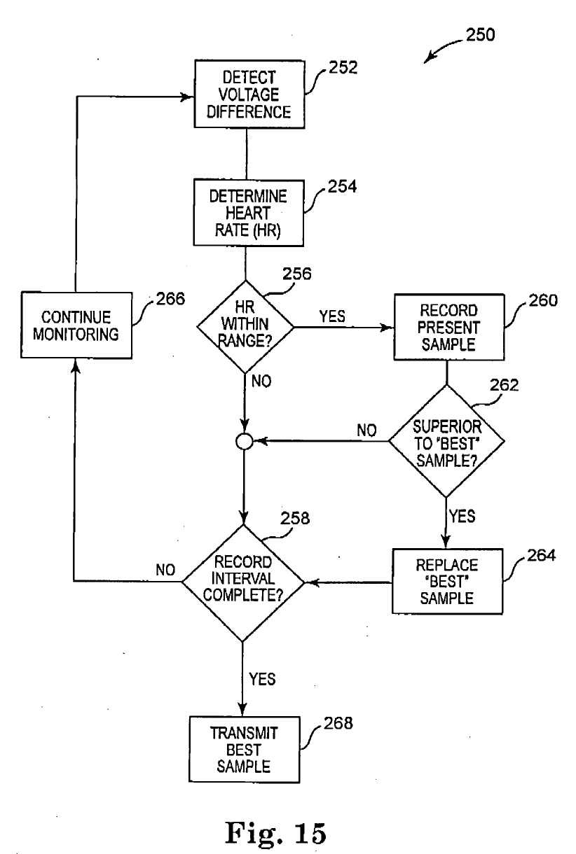

FIG. 15 is a flow diagram illustrating one embodiment of a monitoring and recording process 250 employed by implantable medical device 32. Block 252 illustrates detecting, such as by signal detecting module 236, the voltage difference between conductive housing 102 and remote electrode 106 which, in one embodiment, is representative of the ECG signal of patient 43. At 254, a heart rate of patient 43 is determined, such as by heart rate detector 224. At 256, process 250 queries whether the heart rate is within a predetermined heart rate range. If the answer to the query at 256 is “no”, process 250 proceeds to 258

If the answer to the query at 256 is “yes”, process 250 proceeds to 260, where a current sample segment of the ECG signal is recorded, such as by signal recording module 222. Process 250 then proceeds to 262 where it is queried whether the current sample segment is superior to a best sample segment which was previously recorded. If the answer to the query at 262 is “no”, process 250 proceeds to 258. If the answer to the query at 262 is “yes”, process 250 proceeds to 264, where the previously recorded best sample segment is replaced with the current sample segment.

Process 250 then proceeds to 258, where it is queried whether the recording interval has expired. If the answer to the query at 258 is “no”, process 250 continues monitoring and recording sample segments of the ECG signal, as indicated at 266. If the answer to the query at 258 is “yes”, process 250 proceeds to 268, where the best sample segment for the recording interval is transmitted to remote monitor 40. Process 250 is repeated for each subsequent recording interval.

As described above, in one embodiment, signal processor 42 of monitoring station 40 is configured to measure values of at least one selected characteristic in the recorded sample segments of the ECG signal of patient 43 received from implantable medical device 32. In one embodiment, based on the measured values, signal processor 42 is configured to determine a trend in the selected characteristic of the physiological signal. In one embodiment, based on trend information of the selected characteristic, signal processor 42 is configured to assess a risk of a patient for the occurrence of a physiological event, such as SCD, for example.

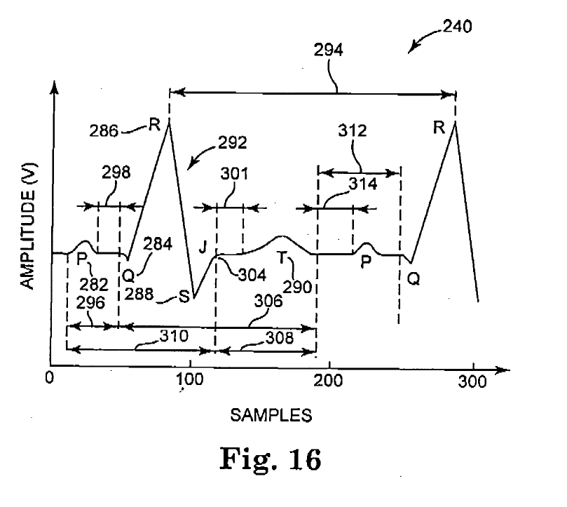

FIG. 16 is a graph illustrating in greater detail the features typically present in a beat 242 of an ECG signal, such as ECG signal 240. As illustrated, each beat, or cycle, 242 typically includes a P-wave 282, a Q-wave 284, an R-wave 286, an S-wave 288, and a T-wave 290. Together, Q-wave 284, R-wave 286, and S-wave 288 form what is commonly referred to as the QRS complex, as indicated at 292. An interval between the apexes of consecutive R-waves, referred to as the R-R interval 294, corresponds to one cycle of the ECG, sometimes referred to as a cardiac cycle or heart beat.

P-wave 282 is associated with the excitation (i.e., depolarization) of the atrial myocardium of the heart. A portion of the ECG from a beginning of P-wave 282 (i.e. onset of atrial depolarization) to a beginning of QRS complex 292 is referred to as the PR-interval, as indicated at 296. A portion from an end of P-wave 282 to an onset of QRS-complex 292 is referred to as the PR-segment, as indicated at 298. PR-segment 298 corresponds to the time between the end of atrial depolarization and the onset of ventricular depolarization.

QRS complex 292 is associated with the excitation (i.e., depolarization) of the ventricular myocardium. T-wave 290 represents the recovery (i.e. repolarization) of the ventricles. A portion of the ECG known as the ST-segment, as indicated at 302, begins at its junction with the end of QRS complex 292, a point referred to as the J-point, as indicated at 304, and ends at the beginning of T-wave 290. ST-segment 302 corresponds to a period of ventricular muscle activity before repolarization.

A portion of the ECG from an onset of QRS-complex 292 to an end of T-wave 290 is known as the QT-interval, as indicated at 306, and is associated with the time of ventricular depolarization and ventricular repolarization. An ST-interval, as indicated at 308, from J-point 304 to an end of T-wave 290, generally represents repolarization of the ventricular myocardium. The term “repolarization component”, as used herein, includes at least one feature or characteristic associated with repolarization, such as ST-interval 308. The term “depolarization component”, as used herein, includes at least one feature or characteristic associated with depolarization, and may for example refer to PR-interval 296 (i.e. atrial depolarization), to QRS complex 292 (i.e. ventricular depolarization), or the combination of PR-interval 296 and QRS complex 292 (i.e. atrial and ventricular depolarization), as indicated at 310.

An inter-beat TQ-segment 312 is defined from an end, or offset point, of a T-wave of a first beat, such as T-wave 290, to an onset of a QRS-complex of a next beat. Similarly, an inter-beat TP-segment 314 is defined from the offset point of a T-wave of a first beat, such as T-wave 290, to an onset of a P-wave of a next beat.

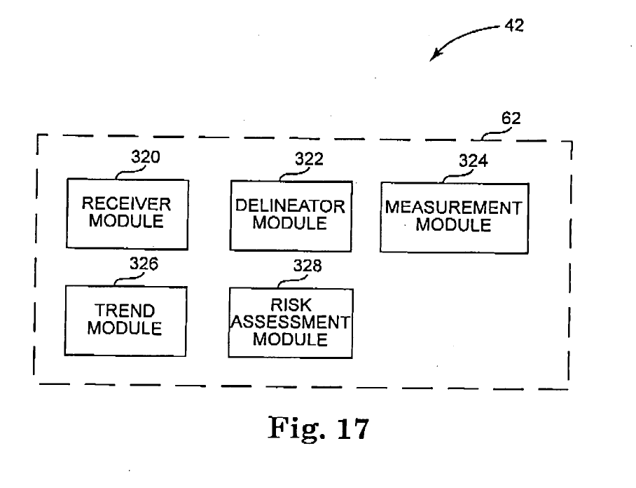

FIG. 17 is block diagram illustrating portions of one embodiment of signal processor 42. Signal processor 42 includes a receiver module 320, a delineator module 322, a measurement module 324, and a trend module 326. In one embodiment, signal processor 42 further includes a risk assessment module 328. In one embodiment, modules 320-328 are included as part of applications block 72 of system memory 62 of computer 42a (see FIG. 3).

In operation, receiver module 320 is configured to receive recorded sample segments of ECG 240 from implantable medical device 32. FIG. 18 illustrates an example of a plurality of recorded sample segments 340 of ECG 240 received from implantable medical device 32, which includes a first recorded sample segment 342, a second recorded sample segment 344, and an n^{th} recorded sample segment 346. As described above, each recorded sample segment of the plurality of recorded samples 340 has a sample duration. In one embodiment, the recorded sample segments may be recorded at predetermined recording intervals over a time period. For example, in one embodiment, each of the recorded sample segments may have a sample duration of 15 seconds and be recorded at 1-week recording intervals over a time period of one or more years. Receiver module 320, in-turn, provides the plurality of recorded sample segments 340 to delineator module 322, the operation of which is described in greater detail below.

With respect to an ECG signal, such as ECG signal 240, beat-to-beat changes in characteristics (e.g. amplitude, duration, shapes, and/or areas) of features (e.g. T-waves, R-R intervals, QT-intervals) may indicate that the heart of patient 43 is electrically unstable and that patient 43 may be at risk of SCD. In some instances, these beat-to-beat changes may manifest themselves in the form of an ABABABAB pattern, where A represents beats having characteristics generally larger in magnitude and B represents beats having characteristics generally smaller in amplitude. Other patterns may occur as well, such as ABCABCABC and ABCDABCD patterns, for example. Such patterns are generally referred to as alternans.

As mentioned briefly above, T-wave alternans (TWA), which is one subset of alternans, has been recognized as a significant indicator of risk for ventricular arrhythmia and SCD. TWA result from different rates of repolarization of the muscle cells of the ventricles of the heart in an ABAB beat pattern. The extent to which these cells non-uniformly recover or repolarize is a recognized basis for electrical instability of the heart and has been associated with a variety of clinical conditions including prolonged QT syndrome, acute myocardial ischemia, and electrolyte disturbance.

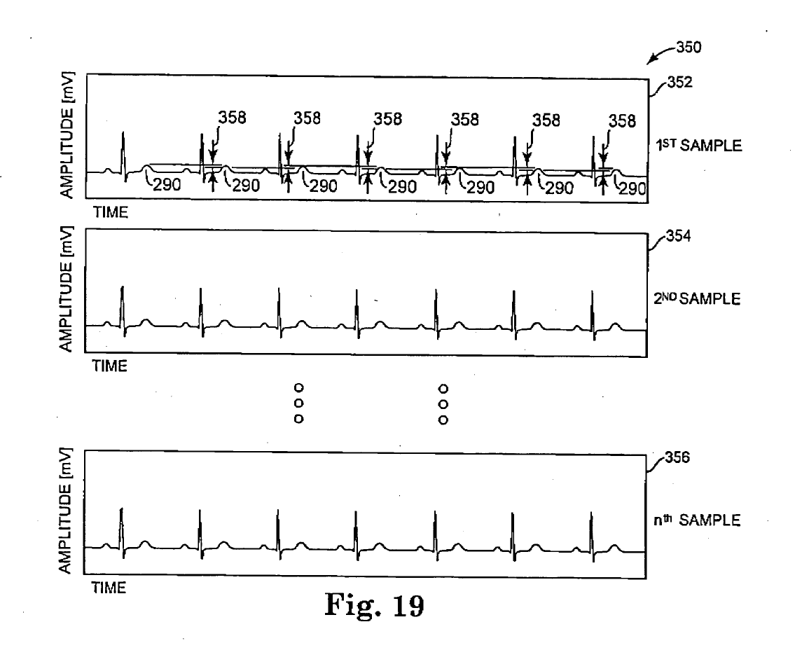

FIG. 19 illustrates an example of a plurality of recorded sample segments 350 of ECG 240 received from implantable medical device 32, including a first recorded sample segment 352, a second recorded sample segment 354, and an n^{th} recorded sample segment 356, wherein the selected characteristic to be measured by system 30 comprises T-wave alternans. Example measurements of the magnitude of T-wave amplitude alternans between consecutive pairs of T-waves 290 are illustrated at 358 with respect to first recorded sample 352.

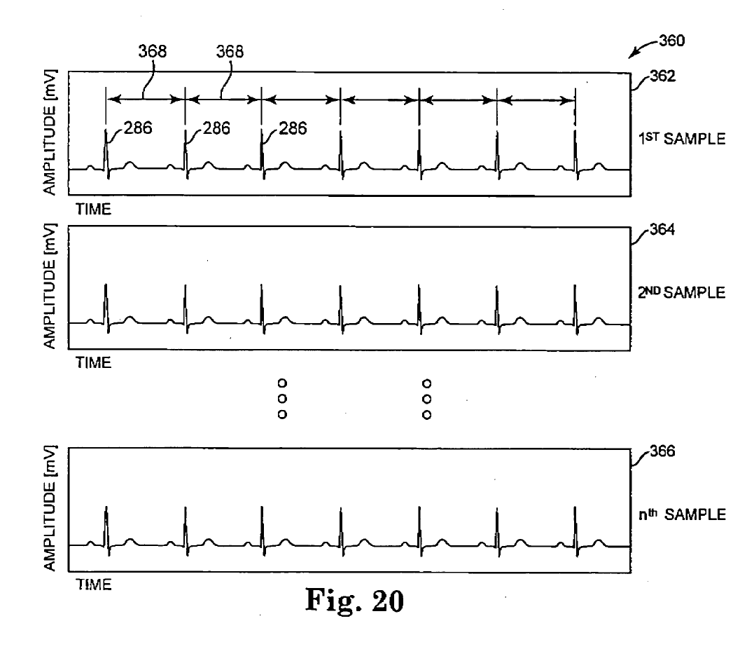

FIG. 20 illustrates an example of a plurality of recorded sample segments 360 of ECG 240 received from implantable medical device 32, including a first recorded sample segment 362, a second recorded sample segment 364, and an n^{th} recorded sample segment 366, wherein the selected characteristic to be measured by system 30 comprises R-R intervals. Example measurements of R-R intervals between consecutive pairs of R-waves 290 are illustrated at 368 with respect to first recorded sample segment 362. R-R intervals 368 may be used to derive characteristics of ECG 240 such as heart rate and heart rate variability.

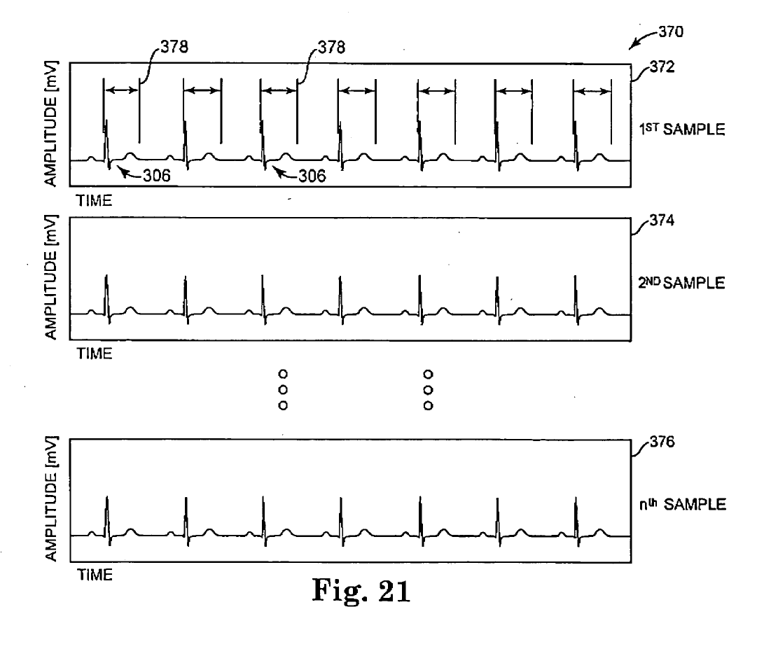

FIG. 21 illustrates an example of a plurality of recorded sample segments 370 of ECG 240 received from implantable medical device 32, including a first recorded sample segment 372, a second recorded sample segment 374, and an n^{th} recorded sample segment 376, wherein the selected characteristic to be measured by system 30 comprises QT-interval alternans. Examples of measurements of QT intervals 306 of beats 242 are illustrated at 378 with respect to first recorded sample segment 372.

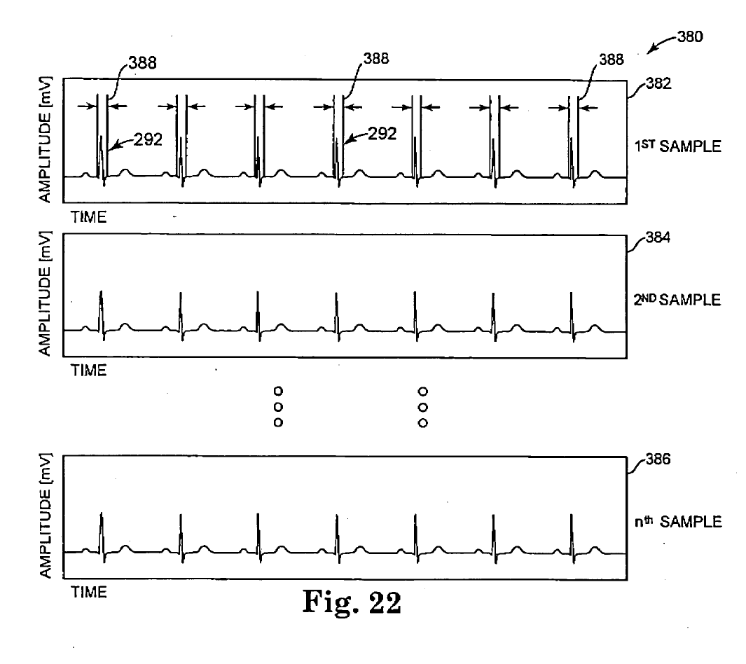

FIG. 22 illustrates an example of a plurality of recorded sample segments 380 of ECG 240 received from implantable medical device 32, including a first recorded sample segment 382, a second recorded sample segment 384, and an n^{th} recorded sample segment 386, wherein the selected characteristic to be measured by system 30 comprises QRS-interval alternans. Example measurements of QRS intervals of QRS-complexes 292 are illustrated at 388 with respect to first recorded sample segment 382.

Delineator 322 receives the recorded sample segments from receiver 320. Delineator 322 is configured to analyze the recorded sample segments to determine a beginning and ending data point of selected features of the ECG signal. In one embodiment, delineator 322 is configured to determine beginning and ending data points of those features required to enable measurement of the at least one selected characteristic of the ECG signal being monitored by system 30. For example, where the selected characteristic comprises T-wave amplitude alternans, delineator 322 determines the beginning and ending data points of each T-wave 290 of each recorded sample segment of the plurality of recorded sample segments 350 (see FIG. 19). In one embodiment, delineator provides only those sub-segments of the recorded sample segments of the ECG signal corresponding to T-waves 290, wherein each data point of the sub-segment includes a time parameter (e.g. sample number) and an amplitude value.

Various techniques may be employed by delineator 322 to determine the beginning and ending data points of features such as, for example, QRS complex detection, QT-interval measurement, and J-point estimation. Such techniques generally determine points of interest in the ECG signal by identifying local and global peaks and valleys, and changes in slope. Another technique employs state and state-transition techniques described by U.S. patent application Ser. No. 11/360,223, filed on Feb. 23, 2006, entitled “System and Method for Signal Composition, Analysis, and Reconstruction.” In the state and state-transition technique, the recorded ECG sample segments are applied to a filter bank having a plurality of filter sections, or component bands. Each component band provides an output signal, or component signal, having a different center frequency, wherein points of interest of the ECG sample segments (e.g. onset points of QRS complexes, R-wave peaks, J-points, etc.) correspond to and can be identified from quarter-phase transition points of the component signals.

Measurement module 324 receives the sub-segments of data points of each of the recorded sample segments of the ECG signal from delineator 322 which correspond to the feature , or features, required to enable measurement of the at least one selected characteristic. Based on these sub-segments of data points, measurement module is configured to measure each occurrence of the at least one characteristic in each recorded sample segment of the plurality of recorded sample segments.

For example, where the selected characteristic comprises T-wave amplitude alternans, measurement module 324 is configured to determine the peak amplitude and corresponding time parameter of each T-wave 290 of each recorded sample segment of the plurality of recorded sample segments 350 of ECG signal 240. In one embodiment, measurement module 324 employs curve-fitting techniques to fit a curve to the series of data points of each sub-segment representing each T-wave 290. The global peak of each fitted curve is found to determine the peak amplitude of the corresponding T-wave 290, and the time parameter is interpolated to determine the corresponding time parameter. The determined peak amplitude of the larger magnitude T-wave of each consecutive pair of T-waves 290 is subtracted from the other T-wave of the pair to determine the corresponding T-wave amplitude alternans 358 (see FIG. 19).

As another example, to measure the QT interval 306 characteristics of each recorded sample segment of ECG 240, measurement module 324 subtracts the time parameter associated with the beginning data point of a sub-segment of data points corresponding to QRS complex 292 from the time parameter associated with the ending data point of the sub-segment of data points corresponding to the subsequent T-wave 290 as received from delineator module 324. Similar techniques are employed to measure other characteristics of ECG 240.

In one embodiment, where the selected characteristic comprises T-wave area alternans, measurement module 324 utilizes curve fitting techniques to fit a curve to each T-wave 290 of each recorded sample segment of the plurality 350 of recorded sample segments of ECG signal 240. The area under the fitted curve is determined as the area of the T-wave and the time parameter corresponding to the peak amplitude of the fitted curve is determined as the corresponding time parameter of the T-wave area measurement.

Trend module 326 receives the measured values of the at least one selected characteristic from measurement module 324 and, based on the measured values, is configured to determine trend information representing a trend in the at least one selected characteristic. In one embodiment, trend module 326 is configured to determine the trend information by performing a modeling operation based on the measured values. In one embodiment, as part of the modeling operation, trend module 326 creates a time-series of data values based on measured values of the at least one selected characteristic and employs time-series analysis (TSA) techniques to determine the trend information. In one embodiment, as a form of TSA, trend module 326 employs curve-fitting techniques to fit a curve to the time-series of data values, wherein in the fitted curve represents a morphology of and is indicative of a trend in the at least one selected characteristic.

In one embodiment, trend module 326 creates the time-series of data values using the “raw” or measured values of the at least one selected characteristic received from measurement module 324. In one embodiment, trend module 326 creates the time-series of data values using composite data values, wherein each of the composite data values is based on a corresponding plurality of the measured values received from measurement module 324.

In one embodiment, trend module 326 forms a single composite data value for each of the recorded sample segments of ECG signal 240. The composite data value may be generated by one of any number of techniques. For example, in one embodiment, trend module 326 forms a single composite value for a recorded sample segment by simply averaging the magnitudes of the measured values of the recorded sample segment. In one embodiment, the composite value for a recorded sample segment comprises a mean value of the measured values of the recorded sample segments. It is noted that the time parameter associated with such a composite value is generated and adjusted accordingly.

In one embodiment, trend module 326 forms a single composite value for more than one of the recorded samples of ECG signal 240. For example, in one scenario, 15-second sample segments of ECG signal 240 may be recorded on a 24-hour recording interval over a time period of a year. In one embodiment, trend module 326 forms a single composite value from the recorded sample segments for each week. As such, trend module forms a single composite value from the seven recorded sample segments for each week and forms a time-series of 52 composite values, one composite value corresponding to each of the 52 weeks of the one year time period.

In one embodiment, trend module 326 forms a single composite value from blocks of N sequential measured values of the at least one selected characteristic, where a block of N sequential measured values may span portions of more than one recorded sample segment of ECG 240. In one embodiment, the composite data value comprises a moving mean of the block of N sequential measured values of the selected characteristic. In one embodiment, the composite value comprises an rms-style average (i.e. an average power). The block of N sequential measured values may be moved one measured value at a time or by some other desired sampling interval. Again, it is noted that the time parameter associated with such composite values is determined and adjusted accordingly.

In one embodiment, trend module 326 is configured to “trim” “outlying” raw or measured values of the at least one selected characteristic when generating a composite value. Any number of techniques may be used for trimming such “outliers.” For example, when a composite value is determined using a block of N sequential measured values, as described above, the N sequential measured values may be sorted or “stacked” by value with a predetermined number of measured values from the top and the bottom of the stack (i.e. highest and lowest values) being removed and not employed in the determination of the corresponding composite value. The trimming could also be based on the number of local standard deviations from the local mean of the block of N sequential measured values. It is noted that such trimming may also be applied to the “raw” or measured values when trend module 326 forms the time-series of data values comprising the “raw” values.

In one embodiment, as described above, after forming the time-series of data values based on the measured values received from measurement module 324, trend module 326 applies TSA techniques to the time-series of data values to determine trend information in the at least one selected characteristic. As also mentioned above, in one embodiment, trend module 326 employs curve fitting techniques as a form of TSA to fit a curve to the time-series of data values, wherein in the fitted curve represents a morphology of and is representative of a trend in the at least one selected characteristic.

Any number of curve-fitting techniques are suitable for use by trend module 326 such as polynomial fitting using the method of least-squares-fit (LSF) to determine coefficients of the polynomial and piece-wise polynomial fitting techniques, for example. Any number of other TSA techniques are also suitable for use by trend module 36 such as auto-regressive moving average (ARMA) techniques, auto-regressive integrated moving average (ARIMA) techniques, and Kalman filtering techniques, to name a few.

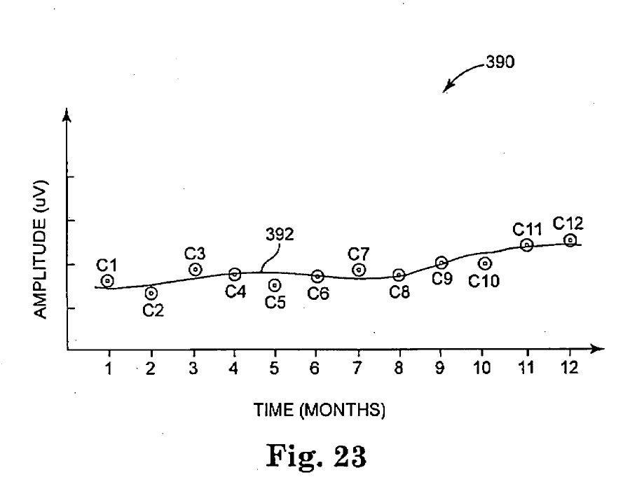

FIG. 23 is a plot 390 illustrating a hypothetical time-series of composite values C1-C12 which are based on measured values of a selected characteristic of ECG 240. In the illustrated illustration of FIG. 23, the selected characteristic comprises T-wave amplitude alternans with time (in months) along the x-axis and amplitude (in micro-volts) along the y-axis. As illustrated, a curve 392 has been fitted to the composite values C1-C12 and is indicative of the morphology and trending of the T-wave amplitude alternans. In the example of FIG. 23, each composite value C1-C12 is a composite of measured T-wave amplitude alternans over a one-month period (i.e. a 1-month recording interval), with composite values C1-C12 spanning a one-year time period.

It is noted that in addition to the time-series and curve-fitting techniques described above, trend module 326 may employ other analysis techniques as well, such as Poincare analysis techniques, for example.

In one embodiment, based on the trend information formed by trend module 326, risk assessment module 328 is configured to evaluate the risk of patient 43 suffering a physiological event for which the measured characteristic is an indicator. For example, as described above, T-wave amplitude alternans has been recognized as being an indicator of ventricular arrhythmia and SCD. As such, in one embodiment, where the at least one selected characteristic comprises T-wave amplitude alternans (or another characteristic indicative of SCD), risk assessment module 328 is configured to assess the risk of SCD to patient 43 based on the trend information provided by trend module 326. In one embodiment, risk assessment module 328 is configured to assign patient 43 to one of a plurality of risk stratification groups for SCD.

In one embodiment, to assess a patient's risk of suffering a physiological event, risk assessment module 328 applies extrapolation techniques to extend the fitted curve determined by trend module 326 to estimate future values (and associated time parameters) of the at least one characteristic. Based on the estimated future values, risk assessment module 238 is able to project and predict a future risk of patient 43 to the physiological event. In one embodiment, risk assessment module 328 is configured to project when the value of the at least one characteristic is likely to exceed a threshold or risk decision value which is indicative of the likelihood of the occurrence of the physiological event. For example, in one embodiment, where the at least one characteristic comprises T-wave amplitude alternans, the risk decision value comprises a predetermined magnitude (e.g. a micro-volt level) of T-wave amplitude alternans which is associated with or indicative of a particular risk level of SCD to patient 43. As such, in one embodiment, by determining when a projected value of T-wave amplitude alternans is likely to exceed the risk decision value, risk assessment module 328 is able to estimate when patient 43 is likely to be at a higher risk of SCD.

In one embodiment, rather than extrapolating to determine a projected future value of the at least one characteristic, risk assessment module 328 is configured to determine a statistic of the fitted curve (e.g. a mean, a trimmed mean, an average, trimmed average, a moving mean based a sampling window, etc.) and compares the statistic to a risk decision value comprising a value of the statistic which is indicative of the likelihood of the occurrence of the physiological event. Similar to that described above, the statistic may also be referred to herein as a “risk value”.

In another embodiment, in lieu of employing curve fitting techniques, risk assessment module is configured to compare the raw or measured data values of the at least one selected characteristic to a risk decision value comprising a value of the at least one characteristic which is indicative of the likelihood of the occurrence of the physiological event. In one embodiment, in lieu of the raw or measured values, risk assessment module is configured to determine a statistic of the measured data values of the at least one selected characteristic (e.g. a mean, a trimmed mean, an average, trimmed average, a moving mean based a sampling window, etc.) and to compare the statistic to the risk decision value.

It is noted that values of the fitted curve, including projected values and statistics, and values of the raw or measured data values, including statistics determined therefrom, may also be referred to herein as a “risk values”. In one embodiment, when the risk value is greater than or equal to the risk decision value, risk assessment module 328 assigns the patient to an “at risk” group, and to a “no risk” group when the risk value is less than the risk decision value. In one embodiment, the risk decision value comprises a range of risk decision values. In one embodiment, risk assessment module 328 assigns the patient to a “high risk” group when the risk value is above the range of risk decision values, to a “medium risk” group when the risk value is within the range of risk decision values, and to a “low risk” group when the risk value is below the range of risk decision values.

In one embodiment, risk assessment module 328 displays the fitted trend curve of the at least one characteristic determined by trend module 326, along with projected values of the at least one characteristic, on a display device, such as display 100. A physician can then assess the risk to the patient based on the displayed information.

Although described above primarily with respect to a single characteristic of ECG signal 240, delineator 322, measurement module 324, and trend module 326 are configured to enable measurement and trending of more than one characteristic of ECG signal 240. In one embodiment, risk assessment module 328 is configured to employ multivariate analysis techniques to correlate a trend of a first selected characteristic with a trend of a second selected characteristic of ECG signal 240 in order to predict the likelihood of the occurrence of a physiological event. For example, risk assessment module 328 may correlate a trend in heart rate variability (see R-R intervals 368 illustrated by FIG. 20) with a trend in t-wave amplitude alternans, as described above, to assess the risk of patient 43 for SCD. Any number of multivariate time-series analysis techniques may be employed by risk assessment module 328 such as multivariate ARMA techniques, multivariate ARIMA techniques, and multivariate Kalman filtering techniques, for example.

In general, the above described TSA techniques, including curve fitting techniques, produce models for the measured values of the at least one characteristic, the models having associated parameters or outputs. In one embodiment, risk assessment module is configured to perform a mapping operation which maps the parameters or outputs to a risk index which reduces the parameters or outputs a single decision variable or risk value. The risk value is then compared to a risk decision threshold or risk decision value, similar to that described above, to assign the patent to one of a plurality of risk groups. In the case of multivariate analysis, the risk decision value comprises a contour in multidimensional space, with the risk value being a point in the multidimensional space.

Examples of mapping techniques suitable for use by risk assessment module 328 include Neural Networking (NN) techniques, Support Vector Machines (SVM), self-organizing maps (SOM), and generative topographic maps (GTM) techniques. It is also noted that, in one embodiment, the NN techniques may operate directly on the raw or measured values of the at least one characteristic to assess the risk to the patient, in lieu of operating on the trend information.

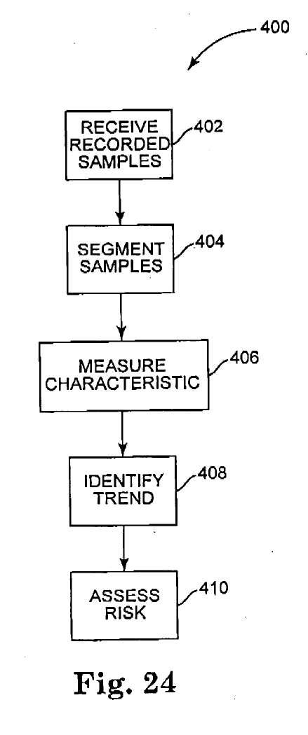

FIG. 24 is a flow diagram illustrating generally one embodiment of a measurement and analysis process 400 employed by signal processor 42. Block 402 illustrates receiving recorded sample segments of ECG signal 240, such as by receiver module 320. At 404, the recorded sample segments are delineated in sub-segments of series of data points corresponding to features of ECG signal 240 necessary to measure the at least one selected characteristic being monitored (e.g. T-wave amplitude alternans), such as described above with respect to delineator module 322.

At 406, the at least one selected characteristic is measured from the sub-segments of each of the recorded sample segments of ECG signal 240, such as described above with regard to measurement module 324. At 408, trends in the at least one selected characteristic are determined, such as through the time-series and curve fitting processes described above with respect to trend module 326. At 410, a risk to a patient for a particular physiological event, for which the at least one selected characteristic is an indicator, is assessed, such as described above with respect to risk assessment module 410.

While described above in detail with particular respect to measuring and analyzing features of an ECG waveform, the embodiments described herein can be applied monitor, measure, and analyze other types of physiological signals, such as a blood pressure signal, for example.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

1. A physiological signal monitoring and analysis system comprising:
an implantable medical device configured to monitor and record sample segments of at least one physiological signal of a patient at time separated recording intervals over a time period; and
a signal processor configured to measure values of at least one selected characteristic of the at least one physiological signal from the recorded sample segments, to determine trend information representing a trend in the at least one selected characteristic based on the measured values, and to assess a risk of a physiological event to the patient based on the trend information.

2. The system of claim 1, wherein the signal processor is configured to determine the trend information by performing a modeling operation based on the measured values.

3. The system of claim 2, wherein the modeling operation is selected from the group consisting of curve fitting techniques, auto-regressive moving average (ARMA) techniques, auto-regressive integrated moving average (ARIMA) techniques, Kalman filtering techniques, and statistical techniques including mean, median, root-mean-square, trimmed mean, and moving-window statistics.

4. The system of claim 1, wherein the trend information comprises the measured values.

5. The system of claim 1, wherein the signal processor is configured to assess the risk by applying neural network techniques to the measured values.

6. The system of claim 1, wherein the signal processor is configured to assess the risk by assigning the patient to one of a plurality of risk stratification groups based on the trend information.

7. The system of claim 1, wherein the signal processor is configured to assess the risk by determining a risk value based on the trend information and to assign the patient to one of a plurality of risk stratification groups based on a comparison of the risk value to a risk decision value.

8. The system of claim 7, where the risk decision value comprises a risk decision value range.

9. The system of claim 7, wherein the signal processor is configured to determine the risk value by performing a mapping operation to map the trend information into a risk index.

10. The system of claim 9, wherein the risk index comprises a multivariate risk index.

11. The system of claim 10, wherein the mapping operation is selected from the group consisting of neural networking (NN) techniques, support vector machine (SVM) techniques, self-organizing mapping (SOM) techniques, generative topographic mapping (GTM) techniques, and fuzzy-neuro techniques.

12. The system of claim 1, wherein the signal processor is configured to assess the risk based on a deviation from a determined trend.

13. The system of claim 1, wherein the time separated recording intervals are selectable.

14. The system of claim 1, wherein the implantable medical device is configured to monitor a heart rate of the patient and to record a sample of the at least one physiological signal when the heart rate is within a predetermined heart rate range.

15. The system of claim 1, wherein the signal processor is configured to form a time-series of data values based on the measured values and to fit a curve to the time-series of data values, wherein the curve is representative of the trend in the at least one selected characteristic.

16. The system of claim 15, where the data values of the time-series comprise the measured values of the at least one selected characteristic.

17. The system of claim 15, wherein each of the data values of the time-series comprises a composite value of a corresponding selected plurality of the measured values of the at least one selected characteristic.

18. The system of claim 15, wherein the signal process is configured to extrapolate from the curve to estimate a future value of the time-series of values.

19. The system of claim 1, wherein the at least one selected characteristic comprises a plurality of characteristics, and wherein the signal processor is configured to measure values of each characteristic of the plurality of characteristics, to determine trend information for each characteristic of the plurality based on the corresponding measured values, and to assess the risk of the physiological event to the patient based on trend information of up to all characteristics of the plurality of characteristics.

20. The system of claim 19, wherein the signal processor is configured to employ multivariate analysis techniques to assess the risk.

21. The system of claim 1, wherein the at least one physiological signal comprises an electrocardiogram signal and the physiological event comprises cardiac arrhythmia.

22. The system of claim 1, wherein the at least one physiological signal comprises an electrocardiogram signal and the at least one selected characteristic is selected from the group consisting of: T-wave amplitude alternans, T-wave area alternans, T-wave duration alternans, QT interval alternans, ST interval alternans, ST segment duration alternans, ST segment elevation alternans, RR interval alternans, R-wave amplitude alternans, R-wave area alternans, R-wave duration alternans, heart rate turbulence, QRS complex duration alternans, QRS complex area alternans, and QRS complex amplitude alternans.

23. A method of monitoring and analyzing at least one physiological signal of a patient, the method comprising:
recording sample segments of at least one physiological signal of a patient at time separated recording intervals over a time period using a medical device implanted within the patient;
measuring values of at least one selected characteristic of the at least one physiological signal from the recorded sample segments;
determining trend information representing a trend in the at least one selected characteristic based on the measured values; and
assessing a risk to the patient of a physiological event based on the trend information.

24. The method of claim 23, wherein determining the trend information includes performing a modeling operation based on the measured values.

25. The method of claim 24, wherein the modeling operation is selected from the group consisting of curve fitting techniques, auto-regressive moving average (ARMA) techniques, auto-regressive integrated moving average (ARIMA) techniques, and Kalman filtering techniques.

26. The method of claim 24, wherein the modeling operation is selected from a group of statistical techniques including mean, median, root-mean-square, trimmed mean, and moving-window statistics.

27. The method of claim 23, wherein the trend information comprises the measured values.

28. The method of claim 23, wherein assessing the risk includes applying neural network techniques to the measured values.

29. The method of claim 23, wherein assessing the risk includes assigning the patient to one of a plurality of risk stratification groups based on the trend information.

30. The method of claim 23, wherein assessing the risk includes:
determining a risk value based on the trend information;
comparing the risk value to a risk decision value; and
assigning the patient to one of a plurality of risk stratification groups based on the comparison.

31. The method of claim 30, wherein the risk decision value comprises a risk decision value range.

32. The method of claim30, wherein determining the risk value includes performing a mapping operation to map the trend information to a risk index.

33. The method of claim 32, wherein the mapping operation is selected from the group consisting of neural networking (NN) techniques, support vector machine (SVM) techniques, self-organizing mapping (SOM) techniques, generative topographic mapping (GTM) techniques, and fuzzy-neuro techniques.

34. The method of claim 32, wherein the risk index comprises a multivariate risk index.

35. The method of claim 23, wherein assessing the risk is based on a deviation from a determined trend.

36. The method of claim 23, wherein the time separated recording intervals are selectable.

37. The method of claim 23, wherein recording sample segments includes:
monitoring the patient's heart rate; and
recording a sample segment of the at least one physiological signal when the heart rate is within a predetermined heart rate range.

38. The method of claim 23, wherein determining the trend includes:
forming a time-series of data values based on the measured values; and
fitting a curve to the time-series, wherein the fitted curve is representative of the trend of the at least one selected characteristic.

39. The method of claim 38, wherein the data values of the time-series comprise the measured values of the at least one selected characteristic.

40. The method of claim 38, wherein each of the data values of the time-series comprises a composite value of a corresponding selected plurality of the measured values of the at least one selected characteristic.

41. The method of claim 38, wherein determining the trend includes extrapolating from the fitted curve to estimate a future value of the time-series of values.

42. The method of claim 23, wherein the at least one selected characteristic comprises a plurality of characteristics, and wherein:
measuring values of the at least one selected characteristic includes measuring values of each of characteristics of the plurality of characteristics from the recorded samples;
determining the trend includes determining trend information representing each of the characteristics of the plurality of characteristics; and
assessing the risk includes correlating the trend information of up to all characteristics of the plurality of characteristics.

43. The method of claim 42, wherein correlating the trend information includes performing a multivariate analysis of the trend information of up to all characteristics of the plurality of characteristics.

44. The method of claim 23, wherein the at least one physiological signal comprises an electrocardiogram signal and the physiological event comprises a cardiac arrhythmia.

45. The method of claim 23, wherein the at least one selected characteristic comprises a T-wave alternans.

46. The method of claim 45, wherein the T-wave alternans is selected from the group consisting of: T-wave amplitude alternans, T-wave area alternans, and T-wave duration alternans.

47. The method of claim 23, wherein the at least one selected characteristic comprises an electrocardiogram signal interval alternans.

48. The method of claim 47, where the electrocardiogram signal interval alternans is selected from the group consisting of: QT interval alternans, ST interval alternans, RR interval alternans, heart rate turbulence, TT interval alternans, and PR interval alternans.

49. The method of claim 23, wherein the at least one selected characteristic comprises an R-wave alternans.

50. The method of claim 49, wherein the R-wave alternans is selected from the group consisting of: R-wave amplitude alternans, R-wave area alternans, and R-wave duration alternans.

51. The method of claim 23, wherein the at least one selected characteristic comprises a QRS complex alternans.

52. The method of claim 51, wherein the QRS complex alternans is selected from the group consisting of: QRS complex duration alternans and QRS complex area alternans.

53. The method of claim 23, wherein the at least one selected characteristic is selected from the group consisting of ST segment elevation.

54. The method of claim 23, wherein the recording intervals are time separated by 24-hours.

55. The method of claim 23, wherein the recording intervals are time separated by one week.

56. The method of claim 23, wherein the time period of one year.

57. A method of assessing the risk of a patient for sudden cardiac death, the method comprising:
recording sample segments of an electrocardiogram signal of the patient at time separated recording intervals over a time period using an implantable medical device implanted within the patient;
measuring values of at least one selected characteristic of the electrocardiogram signal from the recorded sample segments;
determining trend information representing a trend in the at least one selected characteristic based on the measured values; and
assessing a risk to the patient of suffering a cardiac arrhythmia leading to sudden cardiac death based on the trend information.

58. The method of claim 57, wherein determining the trend information includes performing a modeling operation based on the measured values.

59. The method of claim 58, wherein the modeling operation is selected from the group consisting of curve fitting techniques, auto-regressive moving average (ARMA) techniques, auto-regressive integrated moving average (ARIMA) techniques, and Kalman filtering techniques.

60. The method of claim 58, wherein the modeling operation is selected from a group of statistical techniques including mean, median, root-mean-square, trimmed mean, and moving-window statistics.

61. The method of claim 57, wherein the trend information comprises the measured values.

62. The method of claim 57, wherein assessing the risk includes applying neural networking techniques to the measured values.

63. The method of claim 57, wherein assessing the risk includes assigning the patient to one of a plurality of risk stratification groups based on the trend information.

64. The method of claim 57, wherein assessing the risk includes:
determining a risk value based on the trend information;
comparing the risk value to a risk decision value; and
assigning the patient to one of a plurality of sudden cardiac death risk stratification groups based on the comparison.

65. The method of claim 64, wherein the risk decision value comprises a risk decision value range.

66. The method of claim 64, wherein determining the risk value includes performing a mapping operation to map the trend information to a risk index comprising a plurality of risk values.

67. The method of claim 66, where the mapping operation is selected from the group consisting of neural networking (NN) techniques, support vector machine (SVM) techniques, self-organizing mapping (SOM) techniques, generative topographic mapping (GTM) techniques, and fuzzy-neuro techniques.

68. The method of claim 66, wherein the risk index comprises a multivariate risk index.

69. The method of claim 57, wherein assessing the risk is based on a deviation from a determined trend.

70. The method of claim 57, where the time separated recording intervals are selectable.

71. The method of claim 57, wherein recording the sample segments includes:
monitoring the heart rate of the patient; and
recording a sample segment of the electrocardiogram signal when the heart rate is within a predetermined heart rate range.

72. The method of claim 71, wherein the predetermined heart rate range is from 92 beats per minutes to 115 beats per minute inclusive.

73. The method of claim 57, wherein determining the trend information includes:
forming a time-series of data values based on the measured values; and
fitting a curve to the time-series, wherein the fitted curve is representative of the trend of the at least one selected characteristic.

74. The method of claim 73, wherein assessing the risk includes:
extrapolating from the fitted curve to determine a future data value of the time-series;
comparing the future data value to a risk decision value which is indicative of a risk level of suffering a cardiac arrhythmia.

75. The method of claim 73, wherein the data values of the time-series comprise the measured values of the at least one selected characteristic.

76. The method of claim 73, wherein each of the data values of the time-series comprise a composite value of a corresponding selected plurality of the measured values of the at least one selected characteristic.

77. The method of claim 57, wherein the at least one selected characteristic comprises a plurality of characteristics, and wherein:
measuring values of the at least one selected characteristic of the electrocardiogram signal includes measuring values of each of the characteristics of the plurality of characteristics from the recorded samples;
determining the trend includes determining trend information representing each of the characteristics of the plurality of characteristics; and
assessing the risk includes correlating the trend information of up to all characteristics of the plurality of characteristics.

78. The method of claim 77, wherein correlating the trend information includes performing a multivariate analysis of the trend information of the first and second characteristics.

79. The method of claim 57, wherein the at least one selected characteristic is selected from the group consisting of: T-wave amplitude alternans, T-wave area alternans, T-wave duration alternans, QT interval alternans, ST interval alternans, ST segment duration alternans, ST segment elevation alternans, RR interval alternans, R-wave amplitude alternans, R-wave area alternans, R-wave duration alternans, heart rate turbulence, QRS complex duration alternans, QRS complex area alternans, and QRS complex amplitude alternans.
